(19) United States
(12) Patent Application Publication
(10) Pub. No.: US 2016/0015387 A1
(43) Pub. Date: Jan. 21, 2016

(54) INSERTION TOOL AND PUNCTURE APPARATUS

(71) Applicant: TERUMO KABUSHIKI KAISHA, Tokyo (JP)

(72) Inventors: Masakatsu KAWAURA, Sunnyvale, CA (US); Nao YOKOI, Sunnyvale, CA (US); Shigeki ARIURA, Ebina-shi (JP)

(73) Assignee: TERUMO KABUSHIKI KAISHA, Tokyo (JP)

(21) Appl. No.: 14/870,901

(22) Filed: Sep. 30, 2015

Related U.S. Application Data
(63) Continuation of application No. PCT/JP2013/059892, filed on Apr. 1, 2013.

(51) Int. Cl. A61B 17/06; A61B 17/34; A61B 17/062

(52) U.S. Cl. CPC A61B 17/06109; A61B 17/062; A61B 17/3403; A61B 17/3468; A61F 2/0045

(57) ABSTRACT

An insertion tool is disclosed, which includes an elongate vaginal insertion portion configured to be inserted into a vagina; an elongate urethral insertion portion configured to be inserted into a urethra; and a supporting portion configured to support the vaginal insertion portion and the urethral insertion portion thereon. In a mounted state of the insertion tool in which the vaginal insertion portion is inserted in the vagina and the urethral insertion portion is inserted in the urethra, a puncture needle can puncture a vaginal wall from an outside of a body through a space defined by the vaginal insertion portion, the urethral insertion portion and the supporting portion.

### CROSS-REFERENCES TO RELATED APPLICATIONS

This application is a continuation of International Application No. PCT/JP2013/059892 filed on Apr. 1, 2013, the entire content of which is incorporated herein by reference.

### TECHNICAL FIELD

The present disclosure generally relates to an insertion tool and a puncture apparatus.

### BACKGROUND DISCUSSION

If a person suffers from urinary incontinence, for example, from stress urinary incontinence, then urine leakage occurs when abdominal pressure is applied during normal movement or by laughing, coughing, or sneezing. This is caused, for example, by a fact that pelvic floor muscles which are muscles supporting the urethra are loosened by birth.

For the treatment of urinary incontinence, surgical therapy is effective, and for example, a living body tissue supporting strip indwelling called “sling” is used. A sling is indwelled in the body to support the urethra (for example, refer to Japanese Patent Laid-Open No. 2010-99499). In order to indwell the sling in the body, the operator would incise the vagina with a scalpel, peel off a region between the urethra and the vagina and cause the peeled off region and the outside to communicate with each other through an obturator foramen using a puncture needle or the like. Then, in such a state as just described, the sling is indwelled in the body.

However, if the vagina is incised, the sling may be exposed to the inside of the vagina through a wound caused by the incision or complications such as infection from the wound may be caused. Further, since the vagina is incised, there is a drawback that the invasion can be significant and the burden on the patient is relatively high. Further, the urethra or the like may be damaged during the manipulation by the operator, and the operator's finger may also be damaged.

Here, according to the surgical therapy described above, in order to pass a needle tip of the puncture needle between the urethra and the vagina, it is necessary for the urethra and the vagina to be spaced sufficiently from each other. However, depending upon the patient, the urethra and the vagina may not be spaced sufficiently from each other by various causes. In this case, it is necessary to carry out a process of, for example, injecting physiological saline solution, local anesthetic or the like into the living body tissue between the urethra and the vagina using a syringe to inflate the living body tissue between the urethra and the vagina and thereby sufficiently space the urethra and the vagina from each other. Note that the process just described is hereinafter referred to as “humoral peeling off.”

### SUMMARY

An insertion tool and a puncture apparatus are disclosed which can readily carry out humoral peeling off.

An insertion tool is disclosed, which can include an elongate vaginal insertion portion configured to be inserted into a vagina; an elongate urethral insertion portion configured to be inserted into a urethra; a supporting portion configured to support the vaginal insertion portion and the urethral insertion portion thereon; and a space defined by the vaginal insertion portion, the urethral insertion portion and the supporting portion. In a mounted state of the insertion tool in which the vaginal insertion portion is inserted in the vagina and the urethral insertion portion is inserted in the urethra, a puncture needle can puncture a vaginal wall from an outside of a body through the space.

According to an exemplary embodiment of the insertion tool, the vaginal insertion portion and the urethral insertion portion have respective non-insertion portions positioned outside the body in the mounted state and spaced away from each other.

According to an exemplary embodiment of the insertion tool, preferably the non-insertion portions are spaced away from each other by a distance of 10 to 40 mm.

According to an exemplary embodiment of the insertion tool, preferably, for example, a through-hole is provided at the supporting portion such that the puncture needle can be threaded through the through-hole.

According to an exemplary embodiment of the insertion tool, for example, the supporting portion is spaced away from the vaginal wall in the mounted state.

According to an exemplary embodiment of the insertion tool, preferably, for example, the supporting portion and the vaginal wall in the mounted state are spaced away from each other by a distance of 100 mm or less.

According to an exemplary embodiment of the insertion tool, preferably, for example, a portion of the vaginal insertion portion which is positioned in the vagina in the mounted state has a portion inclined with respect to the urethral insertion portion such that a distal end side thereof is spaced away from the urethral insertion portion.

According to an exemplary embodiment of the insertion tool, preferably, for example, the inclined portion is inclined with respect to the urethral insertion portion by an inclination angle of 0 to 45 degrees.

According to an exemplary embodiment of the insertion tool, alternatively, the portion of the vaginal insertion portion which is positioned in the vagina in the mounted state may have a flattened shape crushed in an array direction of the urethra and the vagina.

According to an exemplary embodiment of the insertion tool, preferably, for example, at least one first suction hole for sucking a portion of the vaginal wall on the urethral insertion portion side is provided at a portion of the vaginal insertion portion which is positioned in the vagina in the mounted state.

According to an exemplary embodiment of the insertion tool, preferably, for example, a region in which the first suction hole is formed has a width of 9 to 39 mm.

According to an exemplary embodiment of the insertion tool, preferably, for example, a position detection portion capable of detecting an insertion depth in the vagina is provided at the vaginal insertion portion.

According to an exemplary embodiment of the insertion tool, preferably, for example, at least one second suction hole for sucking a portion of a urethral wall on the vaginal insertion portion side is provided at a portion of the urethral insertion portion which is positioned in the urethra in the mounted state.

According to an exemplary embodiment of the insertion tool, preferably, for example, a region in which the second suction hole is provided is opposed to a region in which the first suction hole is provided.

According to an exemplary embodiment of the insertion tool, preferably, for example, the vaginal insertion portion and the urethral insertion portion are removably mounted.

A puncture apparatus is disclosed, which can include an insertion tool and a puncture needle. The insertion tool can include an elongate first insertion portion configured to be inserted into a vagina, an elongate second insertion portion configured to be inserted into a urethra, a supporting portion configured to support the first insertion portion and the second insertion portion thereon, and a space defined by the first insertion portion, the second insertion portion and the supporting portion. In accordance with an exemplary embodiment, the puncture needle is capable of turning with respect to the insertion tool. When the puncture needle is turned to puncture a living body tissue, a needle tip of the puncture needle passes between the first insertion portion and the second insertion portion.

According to an exemplary embodiment of the puncture apparatus, preferably, for example, at least one first suction hole for sucking a portion of a vaginal wall on the second insertion portion side is provided at a portion of the first insertion portion which is positioned in the vagina in a mounted state, and the puncture apparatus further can include a confirmation mechanism configured to confirm a sucked state of the vaginal wall by the first suction hole.

A method is disclosed of forming a path in a living body, the method comprising: inserting an elongate vaginal insertion portion into a vagina; inserting an elongate urethral insertion portion into a urethra; supporting the vaginal insertion portion and the urethral insertion portion on a supporting portion; and puncturing a vaginal wall with a puncture needle from an outside of the living body through a space defined by the vaginal insertion portion, the urethral insertion portion, and the supporting portion.

With the insertion tool and the puncture apparatus, in the mounted state in which the vaginal insertion portion is inserted in the vagina and the urethral insertion portion is inserted in the urethra, a space can be formed between the vagina insertion portion and a portion of the vaginal wall on the urethral insertion portion side. A syringe can be caused to puncture a region between the urethra and the vagina from the vaginal wall through the space, and humoral peeling off can be readily carried out.

### BRIEF DESCRIPTION OF THE DRAWINGS

FIG. 1 is a perspective view depicting a first embodiment of a puncture apparatus of the present disclosure;

FIG. 2 is a lateral view of the puncture apparatus depicted in FIG. 1;

FIG. 3 is a top plan view depicting a puncture member, which includes the puncture apparatus as depicted in FIG. 1;

FIG. 4 is a top plan view (rear view) depicting a guide portion of a frame provided on the puncture apparatus depicted in FIG. 1;

FIG. 5 is a top plan view (rear view) depicting the guide portion of the frame provided on the puncture apparatus depicted in FIG. 1;

FIG. 6 is a top plan view depicting a fixing portion of the frame provided on the puncture apparatus depicted in FIG. 1;

FIG. 7 is a lateral view of an insertion tool, which includes the puncture apparatus depicted in FIG. 1;

FIG. 8A is a lateral view illustrating a positional relationship between a puncture needle and an obturator foramen (pelvis);

FIG. 8B is a front view illustrating a positional relationship between a puncture needle and an obturator foramen (pelvis);

FIG. 9 is a partial enlarged view of a vaginal insertion member, which includes the insertion tool depicted in FIG. 7;

FIG. 10A is a cross sectional views illustrating an example of a shape of a vaginal wall in a natural state;

FIG. 10B is a cross sectional views illustrating an example of a shape of a vaginal wall in a state in which a vaginal insertion portion is inserted in a vagina;

FIG. 11 is a view depicting an implant for use together with the puncture apparatus depicted in FIG. 1;

FIGS. 12A and 12B are views illustrating an operation procedure of the puncture apparatus depicted in FIG. 1;

FIGS. 13A and 13B are views illustrating an operation procedure of the puncture apparatus depicted in FIG. 1;

FIG. 14 is a lateral view illustrating a relationship between the puncture apparatus and the pelvis in the state depicted in FIG. 13A;

FIGS. 15A and 15B are views illustrating an operation procedure of the puncture apparatus depicted in FIG. 1;

FIG. 16 is a lateral view illustrating a relationship between the puncture apparatus and the pelvis in the state depicted in FIG. 15A;

FIGS. 17A and 17B are views illustrating another operation procedure of the puncture apparatus depicted in FIG. 1;

FIG. 18 is a lateral view of an insertion tool, which includes a puncture apparatus according to a second exemplary embodiment of the present disclosure;

FIG. 19 is a partial enlarged cross sectional view of a vaginal insertion tool provided on an insertion tool of a puncture apparatus according to a third exemplary embodiment of the present disclosure; and

FIG. 20 is a partial enlarged cross sectional view of a vaginal insertion tool provided on an insertion tool of a puncture apparatus according to a fourth exemplary embodiment of the present disclosure.

### DETAILED DESCRIPTION

In the following, an insertion tool and a puncture apparatus of the present disclosure are described in detail on the basis of preferred embodiments thereof depicted in the accompanying drawings.

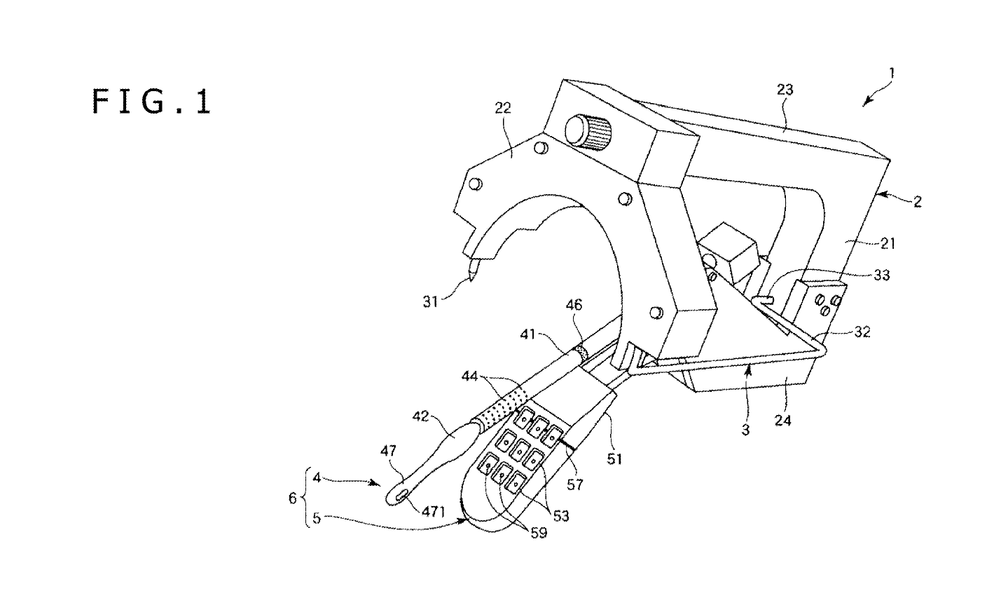

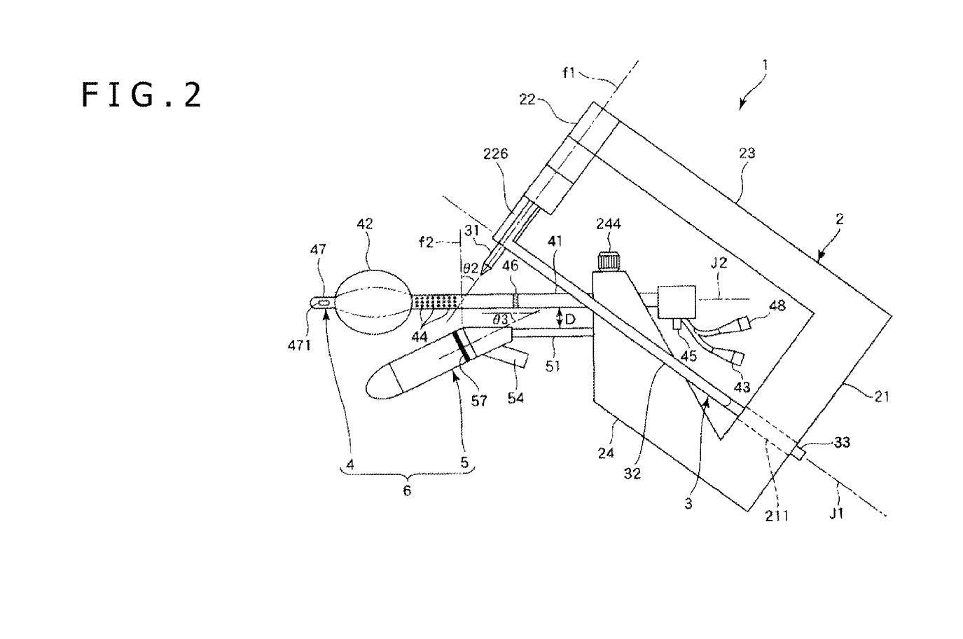

Note that, in the following description, the left side in FIG. 2 is referred to as “distal end,” the right side as “proximal end,” the upper side as “upper,” and the lower side as “lower” for the convenience of description. FIGS. 1 and 2 depict the puncture apparatus in a state in which the puncture apparatus is not used as yet, and in the following description, this state is referred to also as “initial state” for the convenience of description. Further, a state in which the puncture apparatus (insertion tool) depicted in FIG. 1 is mounted on a patient is referred to also as “mounted state.”

The puncture apparatus 1 depicted in FIGS. 1 and 2 is a device that can be used for the treatment of female urinary incontinence, for example, for embedding a living body tissue supporting indwelling for the treatment of urinary incontinence into a living body.

The puncture apparatus 1 can include a frame (supporting member) 2, a puncture member 3, a urethral insertion member 4, and a vaginal insertion member 5. The puncture member 3, the urethral insertion member 4 and the vaginal insertion member 5 are supported on the frame 2. Further, in the puncture apparatus 1, the urethral insertion member 4 and the vaginal insertion member 5 configure an insertion tool 6 suitable for humoral peeling off. In the following, the frame 2, puncture member 3 and insertion tool 6 are described in order.

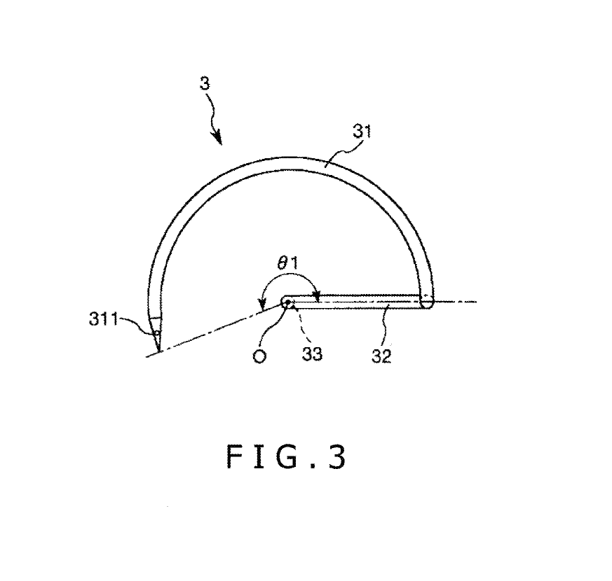

As depicted in FIGS. 1 to 3, the puncture member 3 has a puncture needle 31 for puncturing a living body tissue, a shaft portion 33, and a connection portion 32 for connecting the puncture needle 31 and the shaft portion 33 to each other. Note that the puncture needle 31, connection portion 32 and shaft portion 33 may be formed integrally, or at least one of them may be formed as a separate member from the other members. The puncture member 3 is configured such that it punctures a living body tissue between the urethra and the vagina from the inguinal region (region different from the urethra and the vagina).

In accordance with an exemplary embodiment, the puncture needle 31 has an arcuate shape. The puncture needle 31 has a central angle θ1, which is not limited specifically but is set suitably in accordance with various conditions. However, as hereinafter described, the central angle θ1 is set such that the puncture needle 31 can enter the body from one of the inguinal regions of a patient, pass between the urethra and the vagina and project to the outside of the body from the other inguinal region. For example, the central angle θ1 preferably is 150 to 270 degrees, more preferably is 170 to 250 degrees, and most preferably is 190 to 230 degrees.

A through-hole 311 for holding an implant 9 hereinafter described is formed at a distal end portion of the puncture needle 31.

The shaft portion 33 extends along an axis J1 which crosses with the center O of the puncture needle 31 and crosses orthogonally with a plane f1 which can include the puncture needle 31.

The connection portion 32 connects the proximal end of the puncture needle 31 and the distal end of the shaft portion 33. Further, the connection portion 32 exhibits a substantially L shape bent substantially at the right angle in the middle thereof. The connection portion 32 functions also as a gripping portion to be gripped by an operator when the operator operates the puncture member 3.

The constituent material of the puncture member 3 is not limited especially, but, for example, various metal materials such as stainless steel, aluminum or aluminum alloy, and titanium or titanium alloy can be used.

The frame 2 supports the puncture member 3 for turning motion and allows the urethral insertion member 4 and the vaginal insertion member 5 to be removably fixed thereto. The frame 2 has a function of determining a puncture path of the needle tip of the puncture needle 31 when the puncture needle 31 punctures a living body tissue. In particular, for example, the frame 2 can determine the positional relationship among the puncture member 3, urethral insertion member 4 and vaginal insertion member 5 such that, when the puncture needle 31 punctures the living body tissue, the needle tip of the puncture needle 31 may pass the region between the urethral insertion member 4 and the vaginal insertion member 5 without interfering with any of the urethral insertion member 4 and the vaginal insertion member 5. Consequently, the puncture needle 31 can puncture the living body tissue without puncturing the urethra and the vaginal wall.

As depicted in FIGS. 1 and 2, the frame 2 has a bearing portion 21, which can support the shaft portion 33 of the puncture member 3 for rotation thereon, a guide portion 22 which covers the puncture needle 31 and guides the needle tip of the puncture needle 31, a connection portion 23 which connects the bearing portion 21 and the guide portion 22 to each other, and a fixing portion 24 to which the urethral insertion member 4 and the vaginal insertion member 5 are fixed.

The bearing portion 21 is positioned on the proximal end side of the puncture apparatus 1 and extends in a direction substantially orthogonal to the axis J1. A through-hole 211 is formed on the axis J1 of the bearing portion 21, and the shaft portion 33 is inserted for rotation in the through-hole 211. Consequently, the puncture member 3 is supported for turning motion around the axis J1 on the frame 2.

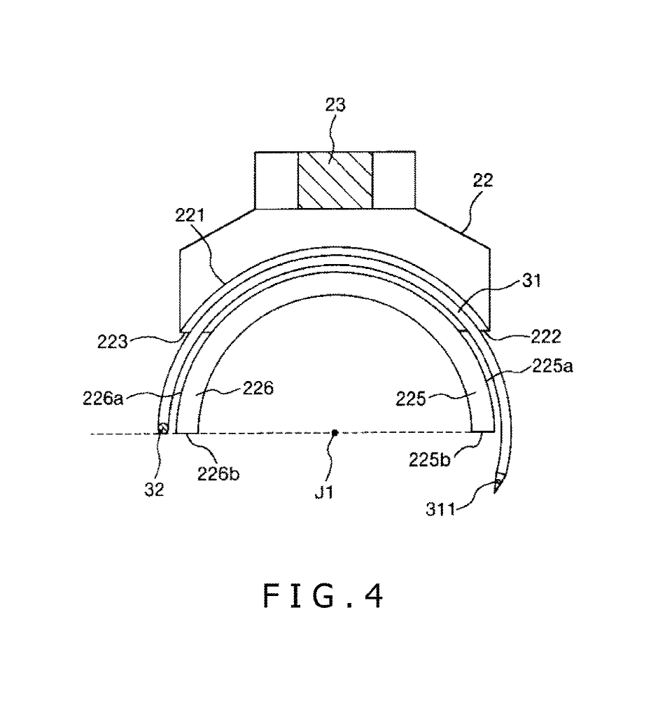

The guide portion 22 is positioned on the distal end side of the puncture apparatus 1 and disposed in an opposing relationship to the bearing portion 21. As depicted in FIG. 4, an arcuate guide groove 221 is formed in the guide portion 22 such that it accommodates and guides the puncture needle 31 in the arcuate guide groove 221. By the guide groove 221, puncture of the puncture needle 31 can be accurately and readily carried out. The overall length of the guide groove 221 is smaller than the overall length of the puncture needle 31, and in an initial state, the needle tip of the puncture needle 31 projects from one end 222 of the guide groove 221 while a proximal end portion of the puncture needle 31 projects from the other end 223.

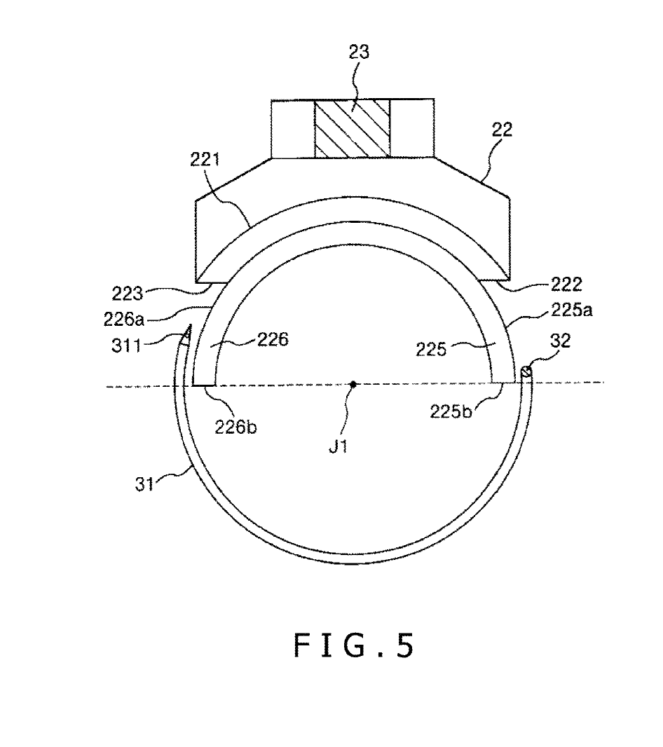

Further, at a portion of the guide portion 22 which is positioned on the inner side with respect to the guide groove 221 (puncture needle 31), a pair of projections 225 and 226 are formed such that they project to the lower side. Of the projections 225 and 226, the projection 225 extends in an arc along the circumference of a distal end portion of the puncture needle 31 while the other projection 226 extends in an arc along the circumference of a proximal end portion of the puncture needle 31. Further, outer side faces 225a and 226a of the projections 225 and 226 are configured from faces continuing to the guide groove 221. In an initial state, the needle tip of the puncture needle 31 can be positioned on the lower side with respect to a lower face 225b of the projection 225 while the proximal end of the puncture needle 31 is positioned at a substantially same height as a lower face 226b of the projection 226. If the puncture needle 31 is turned in the clockwise direction in FIG. 4 from the initial state depicted in FIG. 4, then the puncture needle 31 further projects from the guide groove 221 to be brought into a state depicted in FIG. 5. Note that, depending upon the central angle θ1 of the puncture needle 31, the length of the projection 225 and so forth, the needle tip of the puncture needle 31 may be positioned at the same height as the lower face 225b of the projection 225 or may be positioned on the upper side with respect to the lower face 225b.

The projections 225 and 226 function as pressing portions for pressing the surface of a living body. For example, if the patient has a comparatively thick subcutaneous tissue, then by pressing the projections 225 and 226 against the living body surface, the projections 225 and 226 can be sunk into the living body to allow the puncture needle 31 to puncture the living body deeply. Therefore, the puncture apparatus 1 can cope with a patient whose subcutaneous tissue has a comparatively great thickness.

By forming the projections 225 and 226 on the inner side with respect to the guide groove 221 as in the present embodiment, the puncture needle 31 can be prevented from being hidden by the projections 225 and 226. Therefore, as hereinafter described, a step for threading a string 91 of the implant 9 into the through-hole 311 of the puncture needle 31 can be carried out relatively easily, resulting in improvement of the operability of the puncture apparatus 1.

The connection portion 23 connects the bearing portion 21 and the guide portion 22 to each other. Further, the connection portion 32 is in the form of a bar extending substantially in parallel to the axis J1. The connection portion 32 functions also as a gripping portion, and an operator can grip the connection portion 32 to use the puncture apparatus 1.

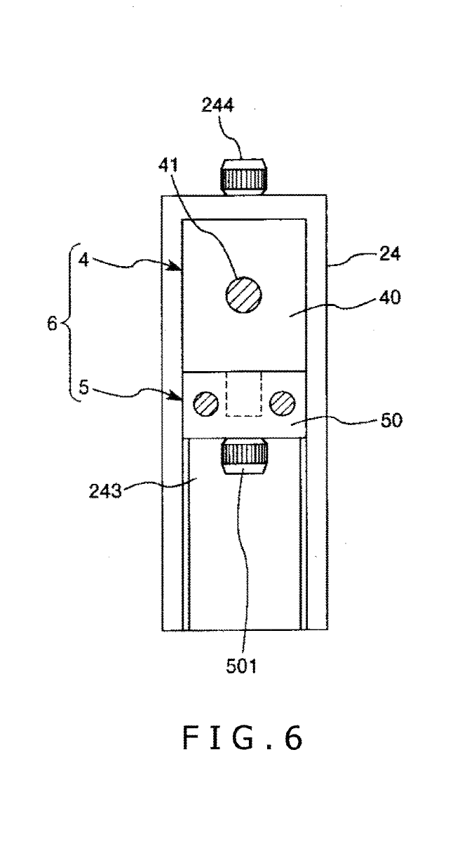

The fixing portion 24 is disposed in an opposing relationship to the connection portion 23 with the axis J1 interposed therebetween. As depicted in FIG. 6, the fixing portion 24 has a recessed portion 243 into which a supporting portion 60 hereinafter described of the insertion tool 6 is to be fitted, and a male thread 244. If the supporting portion 60 is fitted into the recessed portion 243 and besides the male thread 244 is tightened into a female thread (not depicted) of the supporting portion 60, then the insertion tool 6 can be fixed to the fixing portion 24. Note that the configuration of the fixing portion 24 is not limited especially only if the urethral insertion member 4 and the vaginal insertion member 5 can be fixed to the fixing portion 24.

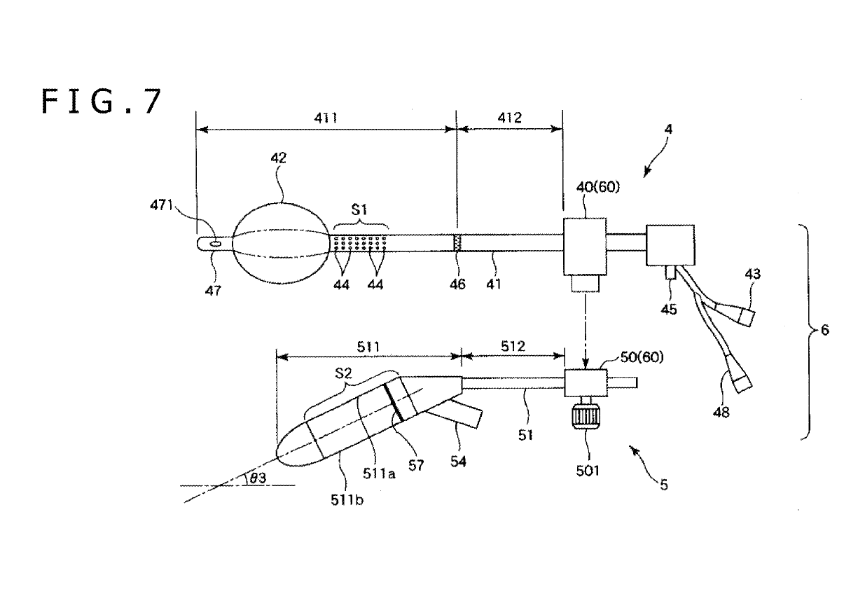

As depicted in FIGS. 1 and 7, the insertion tool 6 has a urethral insertion portion (second insertion portion) 41 which is to be inserted into the urethra, a vaginal insertion portion (first insertion portion) 51 which is to be inserted into the vagina, and the supporting portion 60 which supports the urethral insertion portion 41 and the vaginal insertion portion 51 thereon. As hereinafter described, a space S3 is formed from the vaginal insertion portion 51, urethral insertion portion 41 and supporting portion 60. Such an insertion tool 6 is configured such that, in a mounted state in which the vaginal insertion portion 51 is inserted in the vagina 930 and the urethral insertion portion 41 is inserted in the urethra 920, the puncture needle 31 can puncture the vaginal front wall 931 from the outside of the body through the space S3.

As described hereinabove, the insertion tool 6 is configured from the urethral insertion member 4 and the vaginal insertion member 5, and the urethral insertion member 4 can include the urethral insertion portion 41 while the vaginal insertion member 5 can include the vaginal insertion portion 51. Further, the supporting portion 60 has a supporting portion 40, which is provided on the urethral insertion member 4 and supports the urethral insertion portion 41 and a supporting portion 50 which is provided on the vaginal insertion member 5 and supports the vaginal insertion portion 51. In the insertion tool 6, the urethral insertion member 4 and the vaginal insertion member 5 can be removably mounted through the supporting portions 40 and 50, respectively. By the configuration just described, the operability of the insertion tool 6 can be improved.

In the following, the urethral insertion member 4 and the vaginal insertion member 5 are described in order.

The urethral insertion member 4 has the elongate urethral insertion portion 41 which is inserted to the middle into the urethra, and the supporting portion 40 which supports the urethral insertion portion 41. Note that, in the following description, a portion of the urethral insertion portion 41, which is positioned in the urethra (including the bladder) in the mounted state of the puncture apparatus 1 is referred to also as “insertion portion 411” and another portion of the urethral insertion portion 41, which is exposed to the outside of the body from the urethral orifice in the mounted state and extends to the supporting portion 40 is referred to also as “non-insertion portion 412” for the convenience of description.

The urethral insertion portion 41 has a form of a linear pipe rounded at a distal end thereof. Further, the urethral insertion portion 41 has an inflatable/deflatable balloon 42 and a urine discharge portion 47 provided at a distal end portion of the insertion portion 411 thereof. The balloon 42 functions as a regulation portion for regulating the position of the urethral insertion member 4 in the axial direction in the urethra. In particular, for example, when the puncture apparatus 1 is used, the balloon 42 is inserted into the bladder of the patient and then inflated. Then, the inflated balloon 42 is caught by the bladder neck thereby to fix the position of the urethral insertion member 4 with respect to the bladder and the urethra. Meanwhile, the urine discharge portion 47 is used to discharge urine in the bladder therethrough.

The balloon 42 is connected to a balloon port 43 disposed at a proximal end portion of the urethral insertion portion 41 passing through the inside of the urethral insertion portion 41. A balloon inflation tool such as a syringe can be connected to the balloon port 43. If working fluid (liquid such as saline solution, gas or the like) is supplied from the balloon inflation tool into the balloon 42, then the balloon 42 is inflated, but if the working fluid is taken out from the balloon 42 by the balloon inflation tool conversely, then the balloon 42 is deflated. Note that, in FIG. 7, the balloon 42 in a deflated state is indicated by an alternate long and two short dashes line while the balloon 42 in an inflated state is indicated by a solid line.

A discharge hole 471 is provided in the urine discharge portion 47 such that the discharge hole 471 communicates the inside and the outside of the urine discharge portion 47 with each other. Further, the urine discharge portion 47 passes through the inside of the urethral insertion portion 41 and is connected to a urine discharge port 48 provided at a proximal end portion of the urethral insertion portion 41. Therefore, urine introduced from the discharge hole 471 can be discharged from the urine discharge port 48.

The balloon 42 and the urine discharge portion 47 can be configured, for example, from a double lumen.

Further, a plurality of suction holes (second suction holes) 44 are formed at an intermediate portion of the insertion portion 411 on the proximal end side of the balloon 42. The plurality of suction holes 44 can be disposed over an overall area of the urethral insertion portion 41 in a circumferential direction. Each of the suction holes 44 extends through the inside of the urethral insertion portion 41 and is connected to a suction port 45 provided at a proximal end portion of the urethral insertion portion 41. A suction apparatus such as a pump can be connected to the suction port 45. If the suction apparatus is rendered operative in a state in which the urethral insertion portion 41 is inserted in the urethra, then the urethral wall can be sucked and fixed to the urethral insertion portion 41. Then, if the urethral insertion portion 41 in this state is pushed to the distal end side (into the body), then also the urethra is pushed in together, and for example, the bladder can be displaced to a position which does not overlap with the puncture passage of the puncture needle 31 and the puncture passage of the puncture needle 31 can be relatively assured. Therefore, the puncture of the puncture needle 31 can be carried out relatively accurately and safely. Note that the number of suction holes 44 is not limited particularly and may be, for example, one. Further, the disposition of the suction holes 44 is not limited particularly, and the suction holes 44 may be formed, for example, at only part of the urethral insertion portion 41 in the circumferential direction.

Further, a marker (position detection portion) 46 for allowing confirmation of the insertion depth of the urethral insertion portion 41 in the urethra can be provided at a boundary portion between the insertion portion 411 and the non-insertion portion 412. In accordance with an exemplary embodiment, the marker 46 can be positioned at the urethral orifice when the urethral insertion portion 41 is inserted into the urethra until the balloon 42 is positioned in the bladder. Consequently, the insertion depth of the insertion portion 411 in the urethra can be easily confirmed (detected), resulting in improved operability and safety. As the marker 46, only it is necessary that it be visually recognized from the outside, and the marker 46 can be configured, for example, from a colored portion, a recessed or projected portion. Note that the configuration of the position detection portion is not limited to the marker 46. For example, the marker 46 may be replaced by graduations provided on the surface of the urethral insertion portion 41 and representing the distances from the distal end of the urethral insertion portion 41. With such a position detection portion as just described, the insertion depth of the insertion portion 411 into the urethra can be confirmed more particularly and the operability and the safety are improved further.

The length of the insertion portion 411 is not limited particularly and is set suitably depending upon the length of the urethra, the shape of the bladder and so forth of the patient. Since the length of the urethra can be approximately 30 to 50 mm, the length of the insertion portion 411 preferably can be approximately 50 to 100 mm.

Although the length of the non-insertion portion 412 (spacing distance between the urethral orifice and the supporting portion 40) is not limited particularly, it preferably is equal to or shorter than approximately 100 mm, and more preferably is approximately 20 to 50 mm. Consequently, the non-insertion portion 412 can be made with an appropriate length and is improved in operability. If the length of the non-insertion portion 412 exceeds the upper limit value given above, then depending upon the configuration of the frame 2 and so forth, the center of gravity of the puncture apparatus 1 is displaced by a great distance from the patient, the stability of the puncture apparatus 1 in the mounted state may deteriorate.

The constituent material of the urethral insertion member 4 is not limited particularly, and for example, various metal materials such as stainless steel, aluminum or aluminum alloy, titanium or titanium alloy and so forth or various resin materials can be used.

Here, the inclination angle θ2 (refer to FIG. 2) of the plane f1 with respect to a plane f2 orthogonal to the axis J2 of the urethral insertion portion 41 preferably, for example, is approximately 20 to 60 degrees, more preferably is approximately 30 to 45 degrees, and most preferably is approximately 35 to 40 degrees. In accordance with an exemplary embodiment, for example, the angle defined by the plane f1 and a plane orthogonal to the axis of the urethra preferably, for example, is approximately 20 to 60 degrees, more preferably is approximately 30 to 45 degrees, and most preferably is approximately 35 to 40 degrees. By this, the puncture of the puncture needle 31 can be readily carried out and the puncture distance by the puncture needle 31 can be made shorter.

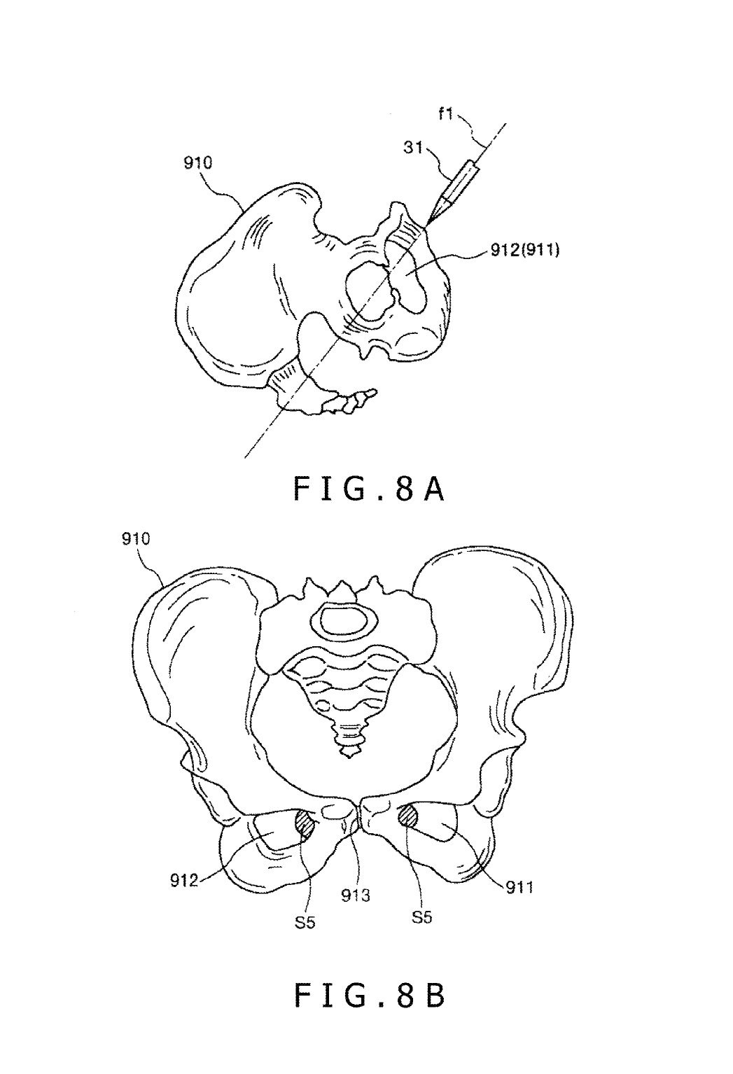

Describing more particularly, by setting the inclination angle θ2 within the range described above, the puncture needle 31 can capture left and right obturator foramens 911 and 912 of a pelvis 910 widely in plane and the puncture space of the puncture needle 31 can be assured wide as depicted in FIG. 8A. In particular, for example, in a state in which the patient is placed in a predetermined position (lithotomy position), the puncture needle 31 can puncture in a comparatively vertical direction with respect to the obturator foramens 911 and 912. Therefore, the puncture of the puncture needle 31 can be readily carried out. In addition, where the puncture needle 31 punctures in a comparatively vertical direction with respect to the obturator foramens 911 and 912, the puncture needle 31 passes a shallow portion of the tissue, and therefore, the needle tip of the puncture needle 31 can pass over a shorter distance between the left and right obturator foramens 911 and 912. Therefore, as depicted in FIG. 8B, the puncture needle 31 can pass the obturator foramens 911 and 912 rather near to a pubic symphysis 913, preferably a safety zone S5. Since the safety zone S5 is a region, which can include a comparatively small number of nerves and blood vessels to which damage is to be avoided, the puncture needle 31 can puncture relatively safely. Therefore, the invasiveness of the procedure can be reduced and the burden on the patient can be relatively low. By setting the inclination angle θ2 to such a range as described above, the puncture of the patient by the puncture needle 31 can be carried out appropriately. Further, by the puncture at the angle specified as above, the tissue between the central urethra which refers to a middle portion of the urethra in the lengthwise direction and the vagina can be determined as a target readily. The position between the central urethra and the vagina is a position suitable as a region into which the implant 9 is to be embedded to carry out the treatment of urinary incontinence.

In contrast, if the inclination angle θ2 is smaller than the lower limit value given hereinabove or is greater than the upper limit value given hereinabove, then depending upon the individual differences of patients, the posture during the procedure, the puncture needle 31 may not capture the obturator foramens 911 and 912 widely in plane or may fail to sufficiently shorten the puncture passage.

More preferably, for example, the puncture needle 31 can easily puncture the region between the central urethra and the vagina if the puncture needle 31 punctures in a state in which the position thereof is displaced such that the urethra, the vagina or both of the urethra and the vagina are pushed into the inner side of the body. The method of pushing one of the urethra and the vagina to the inner side of the body can be, for example, a method of placing the urethral insertion member 4 and/or the vaginal insertion member 5 into a state in which the urethral insertion member 4 and/or the vaginal insertion member 5 are inserted to appropriate positions, absorbing the urethra and/or the vagina through suction holes 44 and 53 provided in the urethral insertion member 4 and/or the vaginal insertion member 5 and then moving the urethral insertion member 4 and/or the vaginal insertion member 5 further to the inner side of the body at predetermined positions along the individual axial lines thereof. In such a manner, in a state in which the position of the puncture needle 31 is displaced such that at least one of the urethra and the vagina is pushed into the inner side of the body, by causing the puncture member 3 to vertically puncture the left and right obturator foramens 911 and 912 of the pelvis 910, a path can be formed at a position suitable for indwelling of the implant 9.

In accordance with an exemplary embodiment, for example, it can be preferable to set the locus of the puncture needle 31 so as to pass the safety zone S5 of the left and right obturator foramens 911 and 912 of the pelvis 910, displace at least one of the urethra and the vagina to the inner side of the body so that the locus may be positioned between the central urethra and the vagina and cause the puncture needle 31 to puncture along the locus to form a path.

As depicted in FIGS. 1 and 7, the vaginal insertion member 5 has the elongate vaginal insertion portion (first insertion portion) 51 which is to be inserted partway into the vagina, and the supporting portion 50 which supports the vaginal insertion portion 51. Note that, in the following description, a portion of the vaginal insertion member 5 to be positioned in the vagina in the mounted state is referred to as “insertion portion 511” and another portion of the vaginal insertion member 5 which is exposed to the outside of the body from the vaginal orifice in the mounted state and extends to the supporting portion 50 is referred to also as “non-insertion portion 512” for the convenience of description.

In accordance with an exemplary embodiment, the insertion portion 511 has a linear shape. Further, the insertion portion 511 extends in an inclined relationship with respect to the insertion portion 411 such that the distal end side of the insertion portion 511 is spaced away from the insertion portion 411. By forming the insertion portion 511 in an inclined relationship with respect to the insertion portion 411, the positional relationship of the insertion portions 411 and 511 can be made closer to the positional relationship between the urethra and the vagina in comparison with those in an alternative case in which the insertion portion 511 is not inclined. Therefore, in the mounted state, the puncture apparatus 1 can be held stably on the patient and the burden on the patient can be reduced. Although the inclination angle θ3 of the insertion portion 511 with respect to the insertion portion 411 is not limited particularly, it preferably is, for example, approximately 0 to 45 degrees, and more preferably is approximately 0 to 30 degrees. By such an inclination angle θ3 as just described, the effect described above can be significantly exhibited. In accordance with an exemplary embodiment, for example, if the inclination angle θ3 is smaller than the lower limit value described above or is greater than the upper limit value described above, then depending upon the individual differences of patients, the posture during the procedure, the vagina or the urethra may be deformed unnaturally in the mounted state and the puncture apparatus 1 may not be held stably.

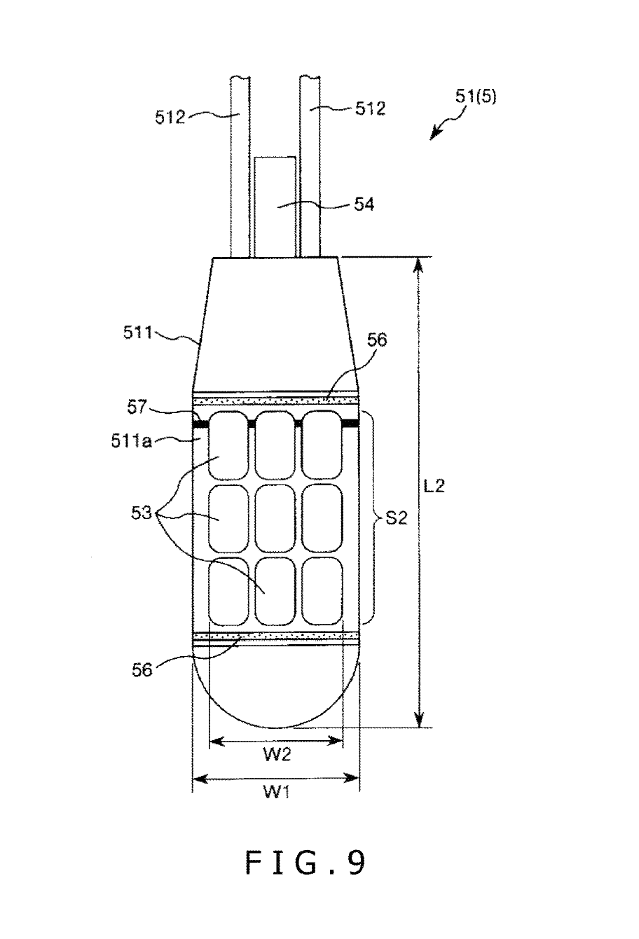

As depicted in FIG. 9, the insertion portion 511 has a flattened shape crushed in the upward and downward direction of the puncture apparatus 1 (array direction of the urethra and the vagina). Further, the insertion portion 511 has a central portion whose width can be substantially fixed and a rounded distal end portion. Although the length L2 of the insertion portion 511 is not limited particularly, for example, it preferably is approximately 20 to 100 mm, and more preferably is approximately 30 to 60 mm. Further, although the width W1 of the insertion portion 511 is not limited particularly, for example, it preferably is approximately 10 to 40 mm, and more preferably is approximately 20 to 30 mm. Further, although the thickness of the insertion portion 511 is not limited particularly, for example, it preferably is approximately 5 to 25 mm, and more preferably is approximately 10 to 20 mm. By forming the insertion portion 511 with such a length×width×thickness as given above, the insertion portion 511 comes to have a shape and a size suitable for general vaginas. Therefore, the stability of the puncture apparatus 1 in the mounted state increases and the burden on the patient can be reduced.

Further, a plurality of suction holes (first suction holes) 53 can be formed on an upper face (face on the urethral insertion portion 41 side) 511a of the insertion portion 511. Note that the number of suction holes 53 is not limited particularly and may be, for example, one. The suction holes 53 are connected to a suction port 54 provided at a proximal end portion of the insertion portion 511 past the inside of the insertion portion 511. The suction port 54 is provided so as to be positioned outside the living body in the mounted state. A suction apparatus such as a pump can be connected to the suction port 54, and if the suction apparatus is rendered operative in a state in which the insertion portion 511 is inserted in the vagina, then the vaginal front wall which is an upper face of the vaginal wall is absorbed and fixed to the insertion portion 511. Then, if the vaginal insertion portion 51 is pushed in to the distal end side (into the body) in a state in which the vaginal wall is absorbed and fixed, then the vaginal wall can be pushed in together with the vaginal insertion portion 51. Therefore, the disposition or the shape of the vaginal wall can be arranged and a puncture passage of the puncture needle 31 can be relatively assured, and the puncture of the puncture needle 31 can be accurately and safely carried out.

A region S2 in which the plurality of suction holes 53 are formed is disposed in an opposing relationship to a region S1. Further, the needle tip of the puncture needle 31 passes between the regions S1 and S2. Since the urethral rear wall which is a lower face of the urethral wall is absorbed to the insertion portion 411 in the region S1 while the vaginal front wall is absorbed to the insertion portion 511 in the region S2, the urethral wall and the vaginal wall are spaced away from each other by a greater distance between the regions S1 and S2. Therefore, by causing the puncture needle 31 to pass through such a region as just described, the puncture needle 31 can be caused to puncture with a relatively high degree of safeness.

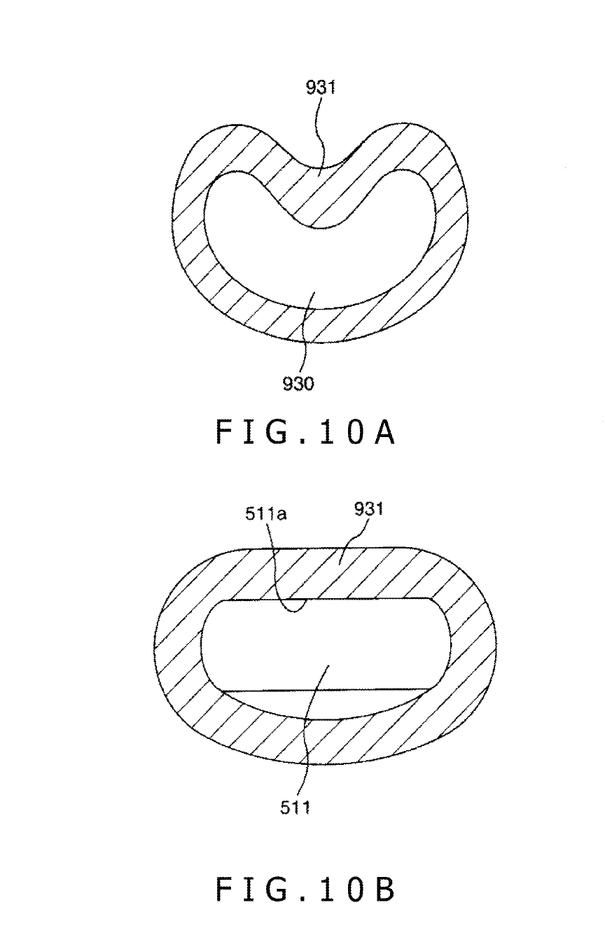

The region S2 spans over a substantially overall area of the upper face 511a in the widthwise direction. Although the width W2 of the region S2 is not limited particularly, for example, it preferably is approximately 9 to 39 mm, and more preferably is approximately 19 to 29 mm. With such a width W2 as just described, the vaginal front wall can be absorbed to the insertion portion 511 with a higher degree of certainty without being influenced much by the shape of the vaginal wall. Particularly, depending upon a patient, a vagina 930 may be formed such that a vaginal front wall 931 thereof partly hangs into the vagina 930 as depicted in FIG. 10A. Also in such a case as just described, if the region S2 has such a width W2 as described above, not only the hanging portion but also portions on the opposite sides of the hanging portion can be absorbed with relative certainty as depicted in FIG. 10B. Therefore, the vaginal front wall can be spaced away from the urethra with a higher degree of certainty without being influenced by the shape of the vagina. Particularly, for example, in the present embodiment, since the insertion portion 511 has a flattened shape, the vaginal front wall can be absorbed so as to be spaced further away from the urethra, and the living body tissue between the urethral wall and the vaginal wall can be further widened.

Further, a marker (puncture position confirmation portion) 57 can be provided on the insertion portion 511 such that the puncture passage of the puncture needle 31 (puncture route of the puncture apparatus 1) can be confirmed therefrom. In particular, for example, the puncture apparatus 1 can be secured such that it can puncture a region between the vaginal wall and the urethral wall which exist on an upper face at a position of the insertion portion 511 at which the marker 57 exists. Therefore, the operability and the safety of the insertion tool 6 can be improved. The marker 57 is provided at least on a lower face 511b of the insertion portion 511. Since the lower face 511b is a face which is directed to the vaginal orifice side in the inserted state and can be confirmed visually by the operator through the vaginal orifice, by providing the marker 57 on the lower face 511b, the puncture route of the puncture needle 31 can be confirmed with a higher degree of certainty. In addition, the insertion depth of the insertion portion 511 in the vagina can be confirmed. Note that only it is necessary for the marker 57 to be viewed from the outside, and the marker 57 can be configured, for example, from a colored portion, a recessed or projected portion or the like.

The non-insertion portion 512 has a form of a thin bar extending substantially in parallel to the urethral insertion portion 41. Further, the non-insertion portion 512 can be spaced away from the non-insertion portion 412 of the urethral insertion portion 41. Although the spacing distance D between the non-insertion portion 512 and the urethral insertion portion 41 is not limited particularly, for example it preferably is approximately 10 to 40 mm in accordance with the spacing distance between the urethral orifice and the vaginal orifice of general women.

Although the length of the non-insertion portion 512 (spacing distance between the vaginal orifice and the supporting portion 50) is not limited particularly, for example, it preferably is equal to or smaller than approximately 100 mm, and more preferably is approximately 20 to 50 mm, which can cause the non-insertion portion 512 to have an appropriate length, and improved operability. If the length of the non-insertion portion 512 exceeds the upper limit value described above, then depending upon the configuration of the frame 2 and so forth, the center of gravity of the puncture apparatus 1 is spaced by a great distance from the patient, and the stability of the puncture apparatus 1 in a mounted state may deteriorate.

The supporting portion 50 has a male thread 501 provided thereon, and the supporting portions 40 and 50 can be fixed to each other by tightening the male thread 501 into a female thread (not depicted) of the supporting portion 40.

The constituent material of the vaginal insertion member 5 is not limited particularly, and, for example, various metal materials such as stainless steel, aluminum or aluminum alloy, titanium or titanium alloy or various resin materials can be used similarly to the vaginal insertion member 4.

The configuration of the puncture apparatus 1 is such as described above.

Note that, while, in the present embodiment, the urethral insertion member 4 and the vaginal insertion member 5 which configure the insertion tool 6 are configured removable, the configuration of the urethral insertion member 4 and the vaginal insertion member 5 is not limited to this, and the urethral insertion member 4 and the vaginal insertion member 5 may be configured non-removable.

Further, while the urethral insertion portion 41 in the present embodiment is fixed to the supporting portion 40, the configuration of the urethral insertion portion 41 and the supporting portion 40 is not limited to this, and the urethral insertion portion 41 may be configured so as to allow selection between a state in which the urethral insertion portion 41 is fixed to the supporting portion 40 and another state in which the urethral insertion portion 41 is slidably movable in the axial direction on the supporting portion 40. In particular, for example, such a configuration may be adopted that, if a male thread provided on the supporting portion 40 is loosened, then the urethral insertion portion 41 is placed into a state in which it can slidably move on the supporting portion 40, but if the male thread is tightened, then the urethral insertion portion 41 is placed into another state in which it is fixed to the supporting portion 40. With the configuration just described, since the length of the non-insertion portion 412 can be adjusted, the convenience in use of the insertion tool 6 is improved. Note that this similarly applies also to the vaginal insertion portion 51.

Further, while the inclination of the axis J1 with respect to the axis J2 (inclination angle θ2) in the present embodiment is fixed, the inclination is not limited to this, and the inclination of the axis J1 with respect to the axis J2 may be variable. If this configuration is applied, then since inclination of the axis J1 can be adjusted in accordance with the patient, the convenience in use of the puncture apparatus 1 is further improved.

In the following, usage of the puncture apparatus 1 is described. However, prior to the description of the usage, the implant 9 for use with the puncture apparatus 1 is described.

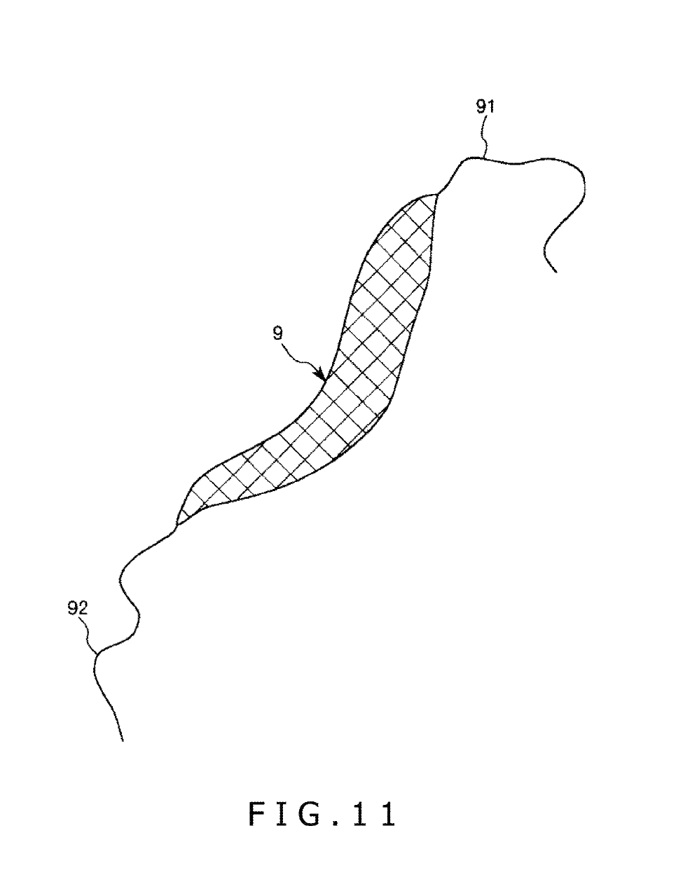

The implant (living body tissue supporting indwelling) 9 depicted in FIG. 11 is a tool which can be embedded for the treatment of urinary incontinence of a female, for example, a tool which supports the urethra, for example, a tool which supports when the urethra tends to move to the vaginal wall side, the urethra so as to restrict the movement of the urethra in a direction away from the vaginal wall. For the implant 9, for example, an elongate article having flexibility can be used.

The implant 9 has a form of a net and has a strip-like general shape. The implant 9 is called “sling.” Note that the implant 9 can be configured from an article formed by crossing linear objects with each other into a braid (lattice shape), for example, a net-like braid. The linear objects may be those having a circular transverse sectional shape, or those having a flattened transverse sectional shape, for example, those of strip-like shape (ribbon shape). To one end of the implant 9, one end portion of the string 91 is fixed, and to the other end portion of the implant 9, one end portion of another string 92 is fixed.

The constituent material of the implant 9 is not limited particularly, and various resin materials, fiber and so forth having biocompatibility such as, for example, polypropylene, polyester elastomer or nylon can be used. Further, the constituent material of the strings 91 and 92 is not limited particularly, and various resin materials, fiber and so forth having biocompatibility such as, for example, polypropylene can be used.

Note that the implant 9 is not limited to such a net-like one as described above only if it can exhibit similar effects.

Now, an operation procedure of the puncture apparatus 1, for example, a procedure when the implant 9 is to be embedded into the living body, is described.

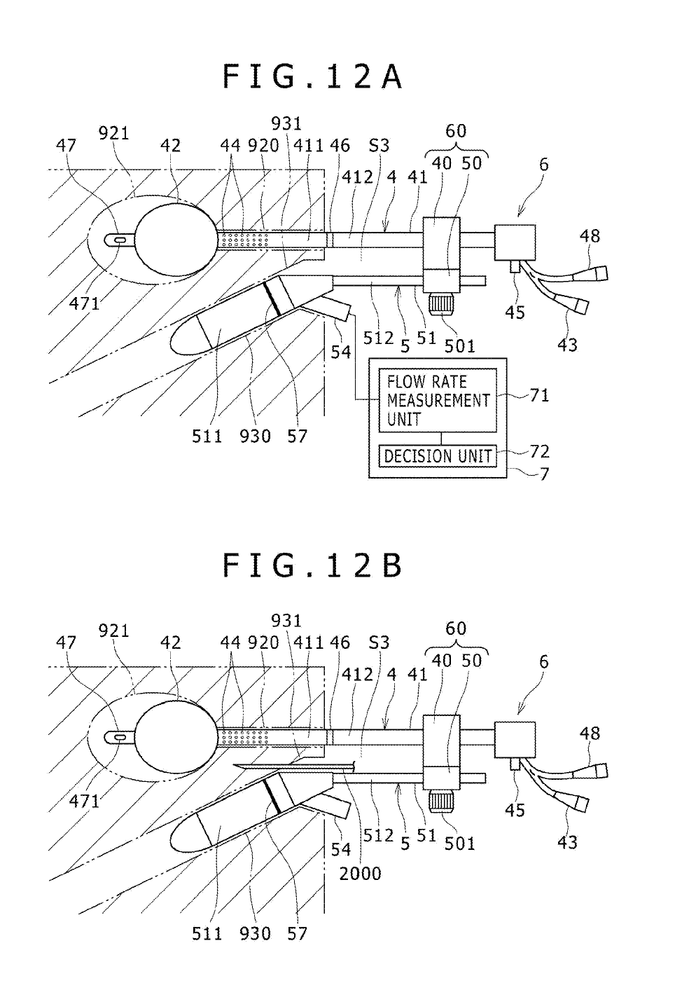

First, a patient is caused to assume a lithotomy position on an operating table, and the insertion tool 6 is mounted on the patient as depicted in FIG. 12A. In particular, a urethral insertion portion 41 of the urethral insertion member 4 is inserted into the urethra 920 of the patient. Thereupon, the insertion depth is confirmed from the marker 46 to dispose the balloon 42 in the bladder 921. The urethra 920 is corrected into a predetermined shape by the predetermined shape urethral insertion portion 41. In the present embodiment, the urethra 920 is corrected to a linear state by the urethral insertion portion 41 of a linear shape.

Then, the balloon 42 is inflated and, as occasion demands, the urine is discharged from within the bladder 921 through the discharge hole 471. Further, the vaginal insertion portion 51 of the vaginal insertion member 5 is inserted into the vagina 930 of the patient. Thereupon, the puncture position of the puncture needle 31 is confirmed from the marker 57 to insert the vaginal insertion portion 51 to an appropriate depth. Then, the male thread 501 is operated to fix the supporting portions 40 and 50. The mounting of the insertion tool 6 on the patient is completed thereby.

In this state, the non-insertion portions 412 and 512 are spaced away from each other and the supporting portion 60 is spaced away from the body surface between the urethral orifice and the vaginal orifice, and the body surface is exposed as depicted in FIG. 12A. In addition, the insertion portion 511 of the vaginal insertion portion 51 and the vaginal front wall 931 are spaced away from each other, and a gap (space) is formed between them. Consequently, a space S3 through which a syringe is to puncture the living body tissue between the urethra 920 and the vagina 930 from the vaginal front wall 931 is formed.

Then, suction apparatuses are connected to the suction ports 45 and 54 and rendered operative to absorb the urethral rear wall to the urethral insertion portion 41 and absorb the vaginal front wall to the vaginal insertion portion 51. For example, if the urethral rear wall is absorbed precisely to the urethral insertion portion 41, then since the suction hole 44 is closed up with the urethral wall, the suction from the suction port 45 is stopped or reduced. Similarly, if the vaginal front wall is absorbed precisely to the vaginal insertion portion 51, then since the suction hole 53 is closed up with the vaginal wall, the suction from the suction port 54 is stopped or weakened. Therefore, from the absorption degree from the suction portions 45 and 54 (for example, from the magnitude of sound generated by the suction), the operator can confirm whether the urethral rear wall and the vaginal front wall are absorbed precisely to the urethral insertion portion 41 and the vaginal insertion portion 51, respectively. From this, it can be considered that the insertion tool 6 has a confirmation mechanism for confirming whether the urethral rear wall and the vaginal front wall are absorbed precisely to the urethral insertion portion 41 and the vaginal insertion portion 51, respectively.

Note that the insertion tool 6 may have a confirmation mechanism 7 for mechanically confirming an absorption state as depicted in FIG. 12A. The confirmation mechanism 7 is not limited particularly only if it can confirm an absorption state. However, the confirmation mechanism 7 can be configured such that it can include, for example, a flow rate measurement unit (negative pressure gauge) 71 for measuring the flow rate at the suction port 54 and a decision unit 72 for deciding on the basis of a result of the measurement from the flow rate measurement unit 71 whether absorption can be precisely carried out.

Then, humoral peeling off is carried out. In particular, for example, the puncture needle of the syringe 2000 punctures the vaginal front wall 931 through the region (space S3) between the insertion portion 511 and the vaginal front wall 931 as depicted in FIG. 12B, and liquid of saline solution or local anesthetic is injected into the living body tissue between the urethra 920 and the vagina 930 (between the regions S1 and S2). Consequently, the living body tissue between the regions S1 and S2 is expanded, and the urethral rear wall is pressed against the urethral insertion portion 41 while the vaginal front wall 931 is pressed against the vaginal insertion portion 51.

Here, preferably the suction from the suction holes 44 and 53 can be carried out continuously also during the humoral peeling off. When the urethral rear wall is pressed against the urethral insertion portion 41 by the humoral peeling off, since the urethral rear wall is further absorbed to the urethral insertion portion 41, the suction from the suction port 45 is stopped or reduced. Similarly, when the vaginal front wall 931 is pressed against the vaginal insertion portion 51, it is further absorbed to the vaginal insertion portion 51, and therefore, the suction from the suction port 45 is stopped or reduced. Accordingly, the operator can confirm from the absorption degrees from the suction portions 45 and 54 whether the humoral peeling off has been precisely carried out.

In order to position the needle tip of the syringe 2000 between the regions S1 and S2, an ultrasonic marker 56 indicative of the position of the region S2 of the insertion portion 511 may be provided (refer to FIG. 9). In FIG. 9, a pair of ultrasonic marker 56 can be provided in such a manner as to sandwich the region S2 between the distal end side and the proximal end side thereof. Consequently, while the operator observes an ultrasonic wave image formed using a transabdominal ultrasonic probe, the operator can position the needle tip of the syringe 2000 between the regions S1 and S2 with certainty. Therefore, humoral peeling off can be carried out with a relative high degree of accuracy. The constituent material of the ultrasonic marker 56 is not limited particularly only if the ultrasonic marker 56 can be visually confirmed on an ultrasonic wave image. In particular, for example, the ultrasonic marker 56 may be configured using various metal materials such as, for example, stainless steel, aluminum or aluminum alloy, titanium or titanium alloy and may have a plurality of fine recessions and projections or the line on the surface thereof. In particular, for example, if a plurality of fine recessions and projections are formed on the surface of a portion configured from a metal material, then the portion at which the recessions and projections are formed can be detected by ultrasonic waves.

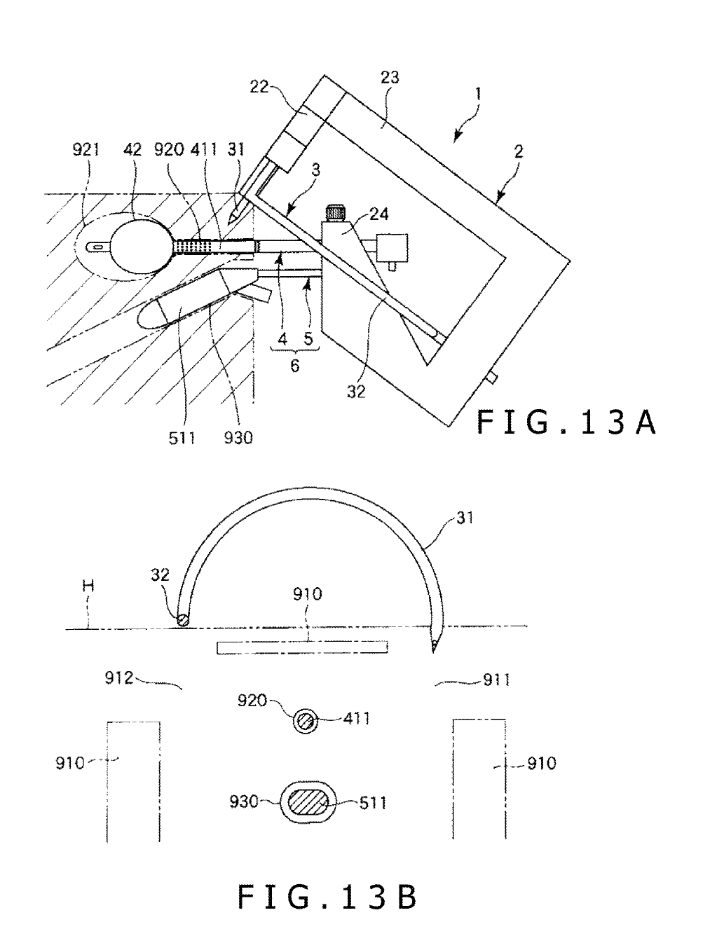

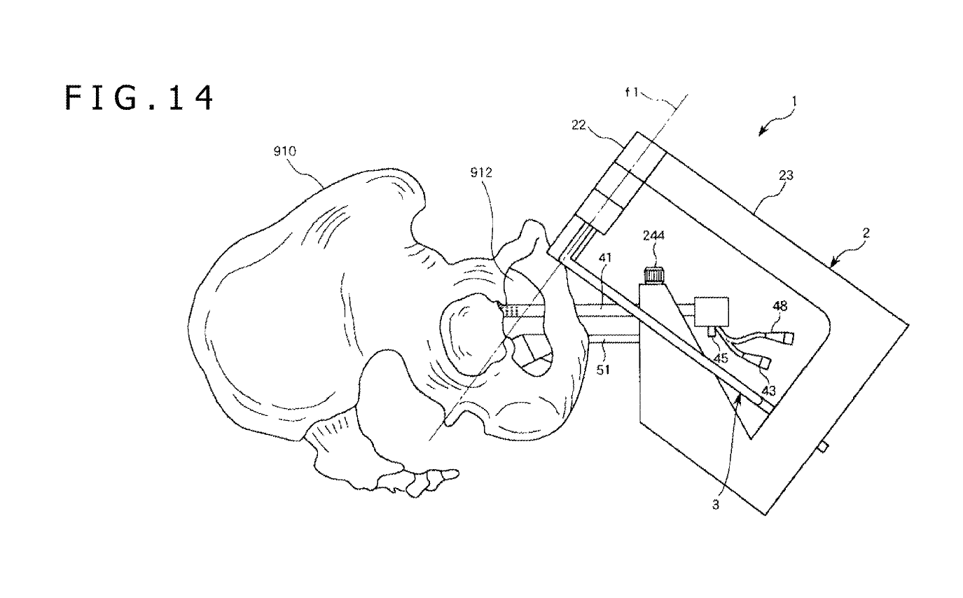

After the humoral peeling off is carried out and the urethral rear wall and the vaginal front wall are placed into a state in which they are spaced away from each other sufficiently, the frame 2 is fixed to the insertion tool 6 as depicted in FIG. 13. Consequently, the puncture apparatus 1 is placed into a state in which it is mounted on the patient. In this state, the positional relationship between the pelvis 910 and the puncture apparatus 1 has such a state as depicted in FIG. 14.

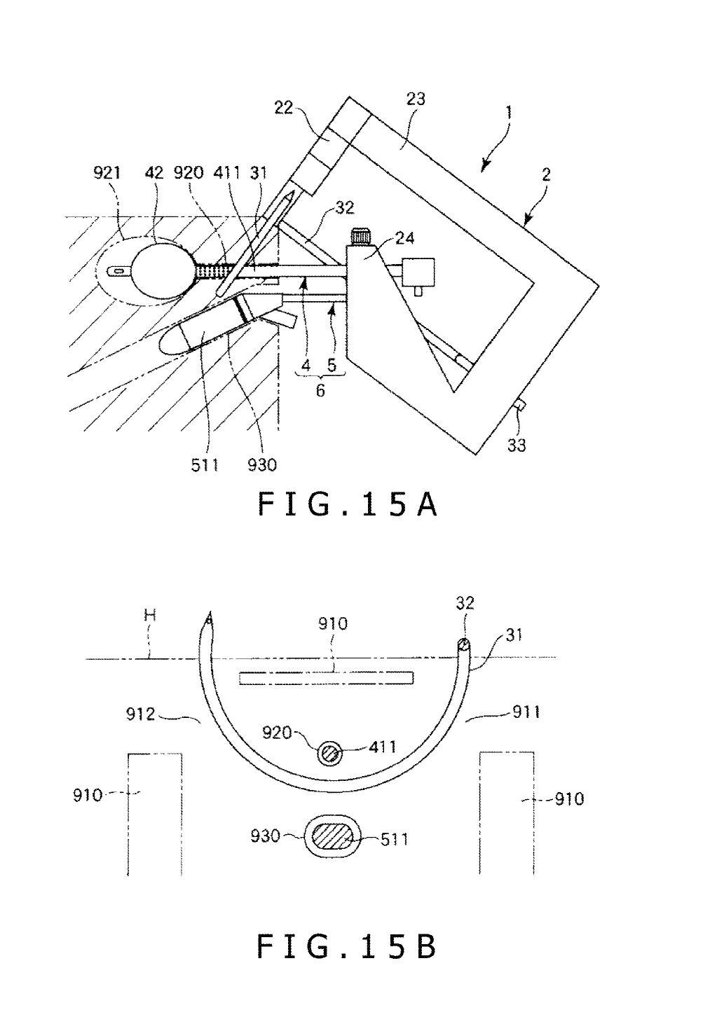

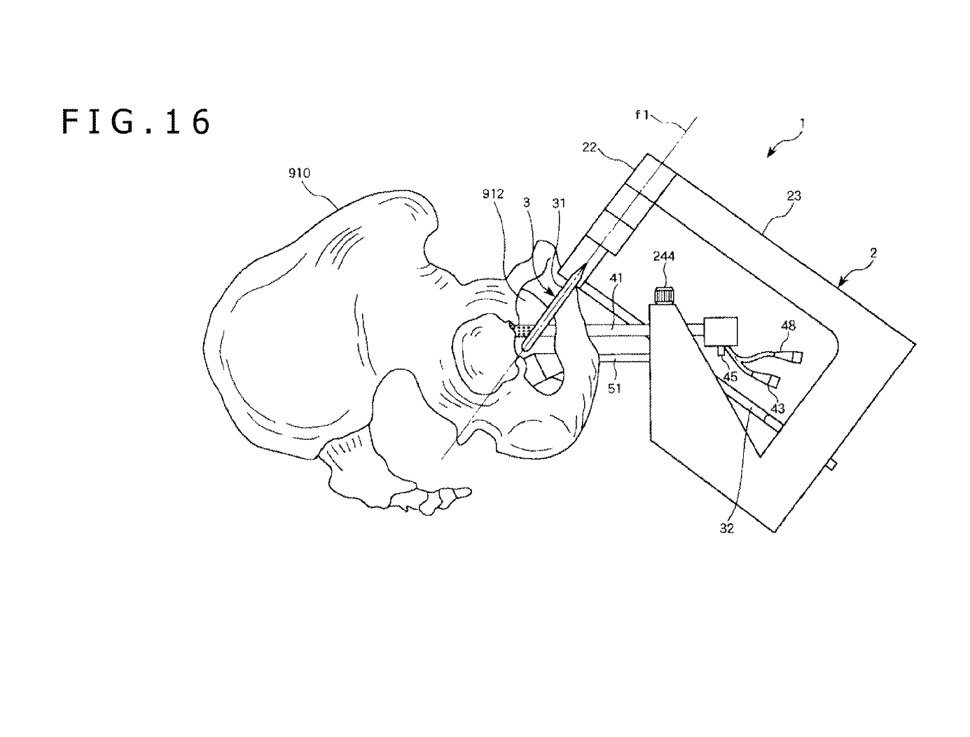

Then, for example, while the connection portion 23 of the frame 2 is gripped by one hand, the puncture member 3 is gripped by the other hand to turn the puncture member 3. Consequently, as depicted in FIGS. 15A and 15B, the needle tip of the puncture needle 31 punctures the body surface H of the left side inguinal region or a location in the proximity of the left side inguinal region of the patient and then enters the inside of the body, passes the left side obturator foramen 911, a region between the urethra 920 and the vagina 930 and the right side obturator foramen 912 in order and projects to the outside of the body from the body surface H at the right side inguinal region or a location in the proximity of the right side inguinal region of the patient. In this state, the positional relationship between the pelvis 910 and the puncture apparatus 1 is such as depicted in FIG. 16. In the puncture apparatus 1, since the insertion portion 511 is inclined such that it is spaced away from the insertion portion 411 as described hereinabove, the space between the urethral wall and the vaginal wall can be relatively assured. Therefore, the puncture of the puncture needle 31 described hereinabove can be carried out with a relative high degree of safety.

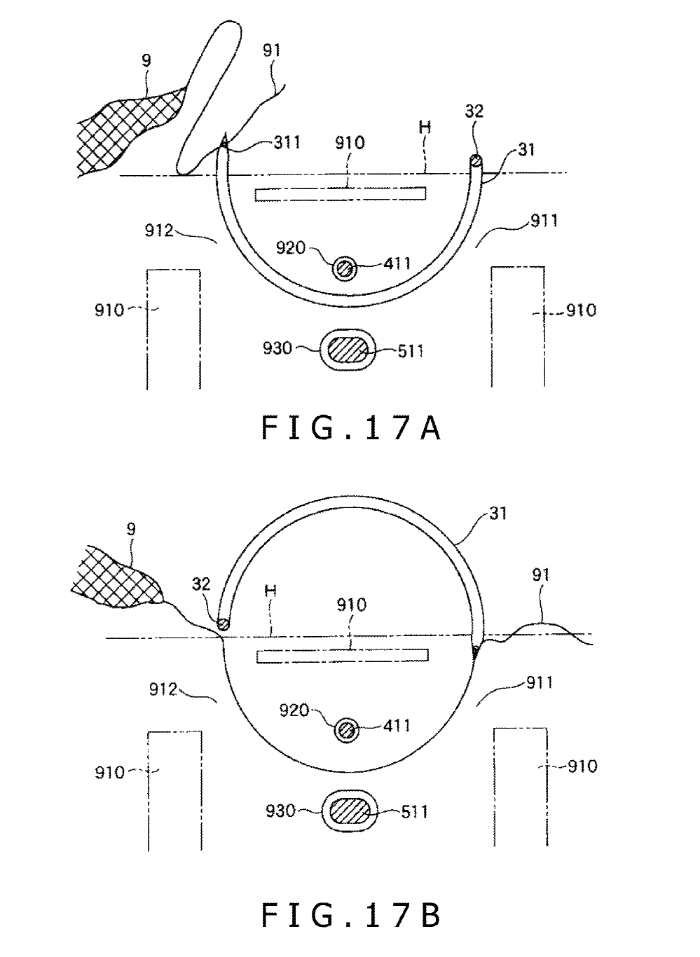

Then, as depicted in FIG. 17A, a string 91 fixed to the implant 9 is threaded into the through-hole 311 of the puncture needle 31 and is held by the puncture needle 31. Then, the puncture member 3 is turned reversely. Consequently, the needle tip of the puncture needle 31 enters the body from the body surface H of the right side inguinal region or a portion in the proximity of the inguinal region of the patient, passes through the obturator foramen 912, the region between the urethra 920 and the vagina 930 and the obturator foramen 911 in order and then goes out from the body surface H in the left side inguinal region or a portion in the proximity of the left side inguinal region of the patent to the outside of the body. In accordance with an exemplary embodiment, for example, the puncture needle 31 can be pulled out to the outside of the body. Then, the string 91 is pulled out from the through-hole 311 and the puncture apparatus 1 is removed from the patient. Then, the string 91 is pulled and the implant 9 is indwelled in the living body.

Thereafter, an unnecessary portion of the implant 9 is cut away, thereby ending the manipulation.

As described above, with the insertion tool 6, since the space S3 can be formed in the mounted state, humoral peeling off can be readily and smoothly carried out. Further, with the puncture apparatus 1, when the implant 9 is to be indwelled, this can be performed with only by a minimally invasive manipulation such as puncture of the puncture needle 31, and the highly invasive incision need not be carried out. [Please confirm that the preceding sentence is accurate]. Therefore, the burden on the patient is relatively light and the safety of the patient is relatively high. Further, the living body can be punctured avoiding the urethra 920 and the vagina 930 by the puncture needle 31, and puncture of the urethra 920 or the vagina 930 by the puncture needle 31 can be safely prevented.

Further, such a situation that the implant 9 is exposed to the inside of the vagina from a wound generated by incision as in the case in which the vagina is incised in a conventional manner or such complications that infection is caused through the wound occur can be prevented. This can be very safe, and the implant 9 can be embedded with relative certainty.

Now, a second exemplary embodiment of an insertion tool and a puncture apparatus of the present disclosure is described.

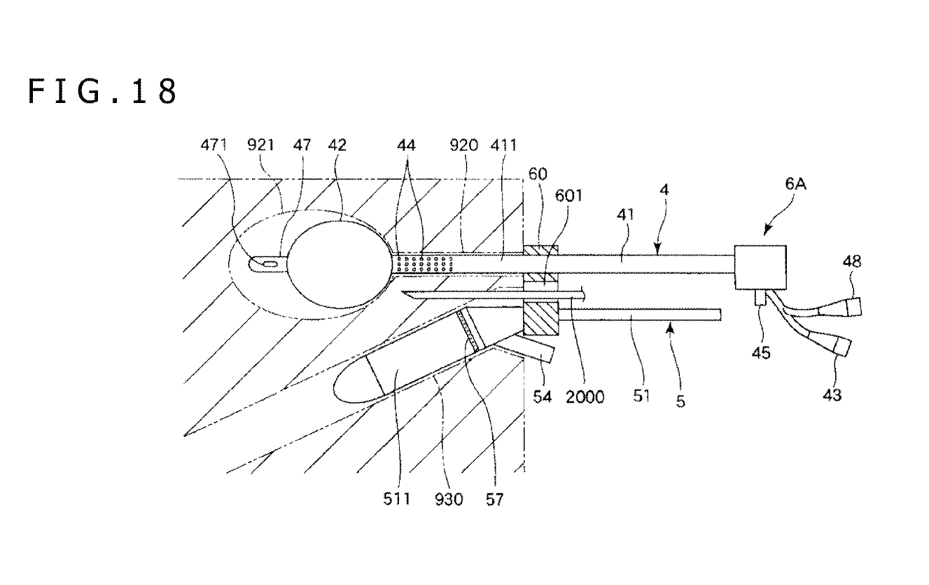

FIG. 18 is a lateral view depicting the insertion tool according to the second exemplary embodiment of the present disclosure.

In the following, description is given principally of differences of the second embodiment from the first embodiment described hereinabove, and description of similar matters is omitted herein.

As depicted in FIG. 18, the insertion tool 6A of the present disclosure contacts, when it is mounted on a patient, at the supporting portion 60 thereof with the living body. In accordance with an exemplary embodiment, for example, both of the non-insertion portions 412 and 512 are omitted. Further, the supporting portion 60 has formed therein a through-hole 601 for threading a syringe 2000 therein. The insertion tool 6A can carry out humoral peeling off by causing the puncture needle of the syringe 2000 to puncture the living body from between the insertion portion 511 and the vaginal front wall through the through-hole 601.

Also with such a second embodiment as described above, similar effects to those achieved by the first embodiment described hereinabove can be achieved.

Now, a third exemplary embodiment of an insertion tool and a puncture apparatus of the present disclosure is described.

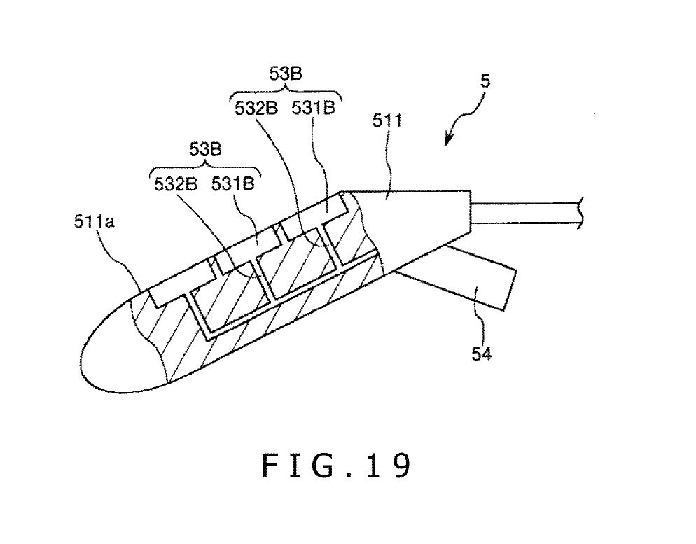

FIG. 19 is a partial enlarged cross sectional view depicting a vaginal insertion tool provided on an insertion tool of the puncture apparatus according to the third embodiment of the present disclosure.

In the following, description is given principally of differences of the third embodiment from the first embodiment described hereinabove, and description of similar matters is omitted herein.

As depicted in FIG. 19, each suction hole 53B is configured such that it is reduced in diameter at an intermediate portion thereof. In particular, for example, each suction hole 53B has a bottomed opening portion 531B open to an upper face 511a and a reduced diameter portion 532B open to a bottom face of the opening portion 531B. Where the suction hole 53B has the reduced diameter portion 532B in this manner, the inside of the suction hole 53B can be efficiently placed into a negative pressure state, and absorption of the vaginal wall can be carried out with relative certainty. In accordance with an exemplary embodiment, for example, even if part of the opening portion 531B is not closed up with the vaginal wall, it is effective in that it can efficiently absorb the vaginal wall.

Also with such a third embodiment as described above, similar effects to those achieved by the first embodiment described hereinabove can be achieved.

Now, a fourth exemplary embodiment of an insertion tool and a puncture apparatus of the present disclosure is described.

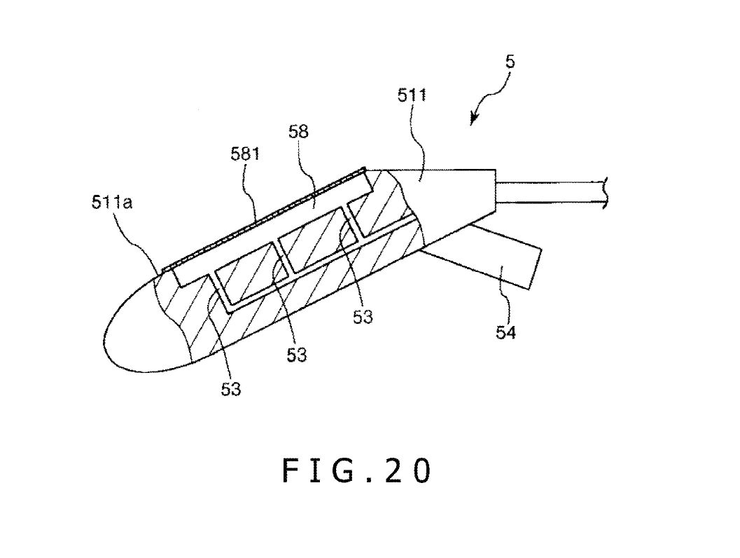

FIG. 20 is a partial enlarged cross sectional view depicting a vaginal insertion tool provided on an insertion tool of the puncture apparatus according to the fourth embodiment of the present disclosure.

In the following, description is given principally of differences of the fourth embodiment from the first embodiment described hereinabove, and description of similar matters is omitted herein.

As depicted in FIG. 20, a bottomed recessed portion 58 is provided on the upper face 511a of the insertion portion 511. The recessed portion 58 has a formation region similar to the region S2 described hereinabove in connection with the first embodiment. Further, a plurality of suction holes 53 are formed on a bottom wall of the recessed portion 58. Further, the opening of the recessed portion 58 is covered with a mesh-like body 581 of a mesh or the like. The mesh-like body 581 can be configured, for example, from an article formed by crossing linear objects with each other into a braid (lattice shape), for example, a net-like braid.

Also with such a fourth embodiment as described above, similar effects to those achieved by the first embodiment described hereinabove can be achieved.

The insertion tool and the puncture apparatus of the present disclosure have been described on the basis of the embodiments depicted in the drawings. However, the present disclosure is not limited to them, and the configuration of the components can be replaced by elements of an arbitrary configuration which have similar functions. Further, the insertion tool and the puncture apparatus may have an arbitrary component added thereto.

Further, while the foregoing description of the embodiments is directed to the configuration by which it can be confirmed on the basis of a suction condition from a suction port whether or not humoral peeling off is carried out precisely, the confirmation may be carried out by some other method. For example, an image pickup element such as a charge coupled device may be disposed in the insertion portion of the vaginal insertion portion such that the confirmation is carried out on the basis of an image obtained from the image pickup device. This similarly applies also to the urethral insertion portion. Alternatively, for example, a lumen into which an endoscope can be inserted may be formed at the vaginal insertion portion such that the confirmation is carried out using the endoscope.

Further, in the embodiments described hereinabove, the second insertion portion which the insertion tool has is a vaginal insertion portion which is inserted into the urethra. However, the second insertion portion is not limited to this and may be a rectal insertion portion which is inserted into the rectum. In accordance with an exemplary embodiment, for example, the insertion tool may have a vaginal insertion portion (first insertion portion) and a rectal insertion portion (second insertion portion). With the insertion portion having such a configuration as just described, when humoral peeling off is carried out for a living body tissue between the vagina and the rectum, effects similar to those achieved by the embodiments described hereinabove can be achieved. For example, when treatment of pelvic organ prolapse (POP) such as hysterocele, vaginal apical prolapse, rectal prolapse or small intestine removed, humoral peeling off is sometimes carried out for the living body tissue between the vagina and the rectum. Therefore, the insertion tool of such a configuration as described above is suitable to carry out treatment of pelvic organ prolapse.

Further, the embodiments described above are directed to a case in which the puncture apparatus of the present disclosure is applied to a device which is used when an implant which can be embedded for the treatment of urinary incontinence of a female is embedded into a living body. However, the application of the puncture apparatus of the present disclosure is not limited to this.

For example, the present disclosure has an application target including excretion failure (such as urinary urgency, frequent urination, urinary incontinence, fecal incontinence, urinary retention and difficulty in urination), pelvic floor disorders including pelvic organ prolapse, vesicovaginal fistula, urethrovaginal fistula, and pelvic pain, which are caused by weakening of the pelvic floor muscles. The pelvic organ prolapse can include such diseases as cystocele, enterocele, rectocele and hysterocele. Alternatively, the pelvic organ prolapse can include such diseases as forward vaginal wall prolapse, rearward vaginal wall prolapse, vaginal apical prolapse and vaginal vault prolapse which are ways to call classified depending upon the prolapsed vaginal wall region.

Further, the hypermobility organizations include the bladder, the vagina, the uterus, intestines and so forth. The fine-moving organizations include bones, muscles, fasciae, ligaments and so forth. Especially, for example, in the pelvic floor disorders, obturator fasciae, coccygeus fasciae, cardinal ligaments, uterosacral ligaments, sacrospinous ligaments and so forth are included.

The manipulations for connecting a hypermobility organization to a fine-moving organization in pelvic floor diseases include a retropubic sling surgery, a transobturator sling surgery (transobturator tape (TOT) surgery), a tension-free vaginal mesh (TVM), a uterosacral ligament suspension (USLS), a sacrospinous ligament fixation (SSLF), a iliococcygeus fascia fixation, a coccygeus fascia fixation and so forth.

According to the present disclosure, an insertion tool can include an elongate vaginal insertion portion configured to be inserted into the vagina, an elongate urethral insertion portion configured to be inserted into the urethra, a supporting portion configured to support the vaginal insertion portion and the urethral insertion portion thereon, and a space defined by the vaginal insertion portion, urethral insertion portion and supporting portion, the insertion tool being configured such that, in a mounted state thereof in which the vaginal insertion portion is inserted in the vagina and the urethral insertion portion is inserted in the urethra, a puncture needle can puncture the vaginal wall from the outside of the body through the space. Therefore, in the mounted state, a gap can be formed between the vaginal insertion portion and the vaginal front wall, and a syringe can be caused to puncture the region between the urethra and the vaginal through the gap from the vaginal front wall and humoral peeling off can be readily carried out.

Accordingly, the insertion tool of the present disclosure has industrial applicability.

The detailed description above describes an insertion tool and a puncture apparatus. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An insertion tool, comprising:
an elongate vaginal insertion portion configured to be inserted into a vagina;
an elongate urethral insertion portion configured to be inserted into a urethra; and
a supporting portion configured to support the vaginal insertion portion and the urethral insertion portion thereon;
wherein, in a mounted state of the insertion tool in which the vaginal insertion portion is inserted in the vagina and the urethral insertion portion is inserted in the urethra, a puncture needle can puncture a vaginal wall from an outside of a body through a space defined by the vaginal insertion portion, the urethral insertion portion and the supporting portion.

2. The insertion tool according to claim 1, wherein the vaginal insertion portion and the urethral insertion portion have respective non-insertion portions positioned outside the body in the mounted state and spaced away from each other.

3. The insertion tool according to claim 2, wherein the non-insertion portions are spaced away from each other by a distance of 10 mm to 40 mm.

4. The insertion tool according to claim 1, wherein a through-hole is provided at the supporting portion such that the puncture needle can be threaded through the through-hole.

5. The insertion tool according to claim 1, wherein the supporting portion is spaced away from the vaginal wall in the mounted state.

6. The insertion tool according to claim 5, wherein the supporting portion and the vaginal wall in the mounted state are spaced away from each other by a distance of 100 mm or less.

7. The insertion tool according to claim 1, wherein a portion of the vaginal insertion portion which is positioned in the vagina in the mounted state has a portion inclined with respect to the urethral insertion portion such that a distal end side of the vaginal insertion portion is spaced away from the urethral insertion portion.

8. The insertion tool according to claim 7, wherein the inclined portion is inclined with respect to the urethral insertion portion by an inclination angle of 0 to 45 degrees.

9. The insertion tool according to claim 7, wherein the portion of the vaginal insertion portion which is positioned in the vagina in the mounted state has a flattened shape crushed in an array direction of the urethra and the vagina.

10. The insertion tool according to claim 1, wherein at least one first suction hole for sucking a portion of the vaginal wall on the urethral insertion portion side is provided at a portion of the vaginal insertion portion, which is positioned in the vagina in the mounted state.

11. The insertion tool according to claim 10, wherein a region in which the first suction hole is formed has a width of 9 mm to 39 mm.

12. The insertion tool according to claim 1, wherein a position detection portion capable of detecting an insertion depth in the vagina is provided at the vaginal insertion portion.

13. The insertion tool according to claim 10, wherein at least one second suction hole for sucking a portion of a urethral wall on the vaginal insertion portion side is provided at a portion of the urethral insertion portion, which is positioned in the urethra in the mounted state.

14. The insertion tool according to claim 13, wherein a region in which the second suction hole is provided is opposed to a region in which the first suction hole is provided.

15. The insertion tool according to claim 1, wherein the vaginal insertion portion and the urethral insertion portion are removably mounted.

16. A puncture apparatus, comprising:
an insertion tool including
an elongate first insertion portion configured to be inserted into a vagina,
an elongate second insertion portion configured to be inserted into a urethra, and
a supporting portion configured to support the first insertion portion and the second insertion portion thereon; and
a puncture needle capable of turning with respect to the insertion tool,
wherein, when the puncture needle is turned to puncture a living body tissue, a needle tip of the puncture needle passes between the first insertion portion and the second insertion portion.

17. The puncture apparatus according to claim 16, wherein at least one first suction hole for sucking a portion of a vaginal wall on the second insertion portion side is provided at a portion of the first insertion portion which is positioned in the vagina in a mounted state.

18. The puncture apparatus according to claim 17, comprising:
a confirmation mechanism configured to confirm a sucked state of the vaginal wall by the at least one first suction hole.

19. A method of forming a path in a living body, the method comprising:
inserting an elongate vaginal insertion portion into a vagina;
inserting an elongate urethral insertion portion into a urethra;
supporting the vaginal insertion portion and the urethral insertion portion on a supporting portion; and
puncturing a vaginal wall with a puncture needle from an outside of the living body through a space defined by the vaginal insertion portion, the urethral insertion portion, and the supporting portion.

20. The method according to claim 19, wherein the vaginal insertion portion and the urethral insertion portion have respective non-insertion portions positioned outside the body; and
spacing apart the non-insertion portions of the vaginal insertion and the urethral insertion portion.
